(19) United States
(12) Patent Application Publication
(10) Pub. No.: US 2009/0299420 A1
(43) Pub. Date: Dec. 3, 2009

(54) METHOD AND APPARATUS FOR CRYOTHERAPY AND PACING PRECONDITIONING

(76) Inventors: Allan C. Shuros, St. Paul, MN (US); Craig Stolen, New Brighton, MN (US); Shantha Arcot-Krishnamurthy, Vadnais Heights, MN (US); Eric A. Mokelke, White Bear Lake, MN (US)

Correspondence Address: SCHWEGMAN, LUNDBERG & WOESSNER/BSC-CRM, PO BOX 2938, MINNEAPOLIS, MN, 55402, US

(21) Appl. No.: 12/474,448

(22) Filed: May 29, 2009

Related U.S. Application Data
(60) Provisional application No. 61/058,089, filed on Jun. 2, 2008.

(51) Int. Cl. A61B 18/02; A61N 1/362

(52) U.S. Cl. 607/3; 606/21

(57) ABSTRACT

A method and apparatus are described for delivering myocardial pacing and cryotherapy in conjunction with a coronary revascularization procedure using one catheter/stent system. In one embodiment, a balloon-stent delivery platform incorporates pacing electrode(s) on or near the distal tip for myocardial pacing.

### CROSS REFERENCE TO RELATED APPLICATIONS

This application claims the benefit of U.S. Provisional Application No. 61/058,089, filed on Jun. 2, 2008, under 35 U.S.C. §119(e), which is hereby incorporated by reference.

This application is related to U.S. patent application Ser. No. 11/113,828 (U.S. Patent Publication No. 20060241704), filed Apr. 25, 2005, hereby incorporated by reference in its entirety and referred to herein as the '828 application.

### FIELD OF THE INVENTION

This invention pertains to apparatus and methods for the treatment of heart disease by revascularization therapies.

### BACKGROUND

When a blood vessel such as the coronary artery is partially or completely occluded, a revascularization procedure such as PTCA (percutaneous transluminal coronary angioplasty) can be performed to reopen the occluded vessel. However the revascularization procedure itself involves a temporary occlusion of the coronary artery. In addition, plaques dislodged and displaced by the revascularization procedure may cause distal embolization where emboli can enter small arteries branching from the original occluded artery and cause occlusion of these branching arteries. This is sometimes referred to the snow plow effect. It is common for patients undergoing reperfusion therapy to often times have cardiac specific enzyme rises following these procedures which indicates tissue damage occurring. The reperfusion process itself can be harmful due to the release of free oxygen species into the blood stream feeding the sensitive myocardium. Besides oxidative stress, reperfusion injury may also result from cytokine release, leukocyte accumulation (neutrophil migration and activation), and calcium overload. This potentially leads to lethal ventricular arrhythmias and poor outcome. Another problem for the PTCA procedure is the occurrence of restenosis in the original treated lesion. Coated stents are routinely used today and have substantially reduced the rate of restenosis, but complications such as late thrombosis and edge of the stent restenosis sometimes occur.

### BRIEF DESCRIPTION OF THE DRAWINGS

FIG. 1 is a diagram of an apparatus for performing a revascularization procedure.

FIG. 2 depicts an exemplary balloon catheter for delivering pacing therapy and cryotherapy.

### DETAILED DESCRIPTION

Cardiac protection pacing in conjunction with coronary revascularization procedures has been described in the above-referenced '828 application. Myocardial pacing has been shown to provide a global cardiac conditioning benefit when applied during and for a short period following reperfusion. Cryotherapy has been implemented in numerous applications to attenuate inflammation. Cooling an atherosclerotic lesion for a period of time induces an apoptotic process to attenuate further inflammation. Described herein is a method and apparatus for delivering myocardial pacing and cryotherapy in conjunction with a coronary revascularization procedure using one catheter/stent system. In one embodiment, a balloon-stent delivery platform incorporates pacing electrode(s) on or near the distal tip for myocardial pacing. Alternatively the stent itself may be the pacing electrode. Cardiac pacing provides a global cardiac conditioning benefit during the reperfusion process. In addition, the catheter includes a heat-sink apparatus to drain thermal energy (cool) from the distal tip to outside the body. The cooling apparatus may comprise a thermoelectric (i.e., Peltier) module for energy transfer. Alternatively, zirconate titanate (PZT) material may be incorporated on the distal aspect of the catheter or the stent itself. When electrically stimulated, the PZT material acts as a heat sink and cools the tissue adjacent to the stent. The objective is to cool the tissue and promote apoptosis without freezing the tissue and inducing necrosis. In one embodiment, the intimal tissue is cooled to around 10 degrees C. for 10-20 seconds to accomplish this goal. Cooling the lesion and the artery in this manner attenuates or prevents further inflammation and restenosis.

An exemplary apparatus includes a catheter adapted for insertion into a coronary artery and an instrument at the distal portion of the catheter for performing a coronary revascularization procedure. The revascularization instrument may be, for example, an inflatable balloon for performing an angioplasty procedure (and possibly stent placement), an atherectomy instrument, or a drug delivery port for infusing a thrombolytic agent. The revascularization instrument may also include a stent delivery platform. The catheter also includes one or more pacing electrodes incorporated into the distal portion of the catheter and a conductor within the catheter for connecting the pacing electrode to pulse generation circuitry. In an embodiment where the catheter is used to deploy a stent, the stent may be used as a pacing electrode.

A heat sink is incorporated into the distal portion the catheter for cooling vascular tissue adjacent the catheter. The heat sink may be a thermoelectric (i.e., Peltier) cooling module and the apparatus further includes a conductor incorporated into the catheter for transmitting power to the thermoelectric cooling module. In another embodiment, the heat sink is a zirconate titanate module and the apparatus further includes a conductor incorporated into the catheter for electrically activating the zirconate titanate. In another embodiment, the heat sink apparatus is a lumen for channeling coolant material to the distal tip. The coolant may be channeled to the distal to the distal tip and expelled or may be circulated in closed-loop fashion. In one embodiment, the coolant is nitrous oxide, and the balloon is filled with liquid nitrous oxide which evaporates into a gas upon entering the balloon. This causes the balloon to expand and cool to approximately −100 C (140 F). Cooling by any of the above-mentioned embodiments is believed to prompt several physiological reactions that open the artery, while doing less damage than standard interventional therapies. The plaque clogging the artery cracks when it freezes, allowing for a more uniform dilation of the blood vessel than occurs in a standard angioplasty. The cooling also prompts apoptosis (programmed cell death), a natural occurrence that minimizes the growth of new tissue (scar), which occurs in response to injury caused by the stretching and tearing of the vessel wall that occurs with conventional therapies.

The pulse generation circuitry used to generate pacing pulses may be configured to deliver pacing pulses in an asynchronous pacing mode. In another embodiment, the apparatus further includes pulse generation and sensing circuitry configured to deliver pacing pulses in a synchronous pacing mode, where the pacing electrode is also used as a cardiac sensing electrode. The apparatus may further include a temperature sensor incorporated into the catheter for sensing the temperature of the tissue cooled by the heat sink and may also include control circuitry for controlling the operation of the heat sink in accordance with the temperature sensed by the temperature sensor.

In an exemplary method for performing a coronary revascularization procedure, a catheter such as described above in introduced into a coronary artery. The catheter may be a conventional over-the-wire type in which the catheter is advanced through the vasculature over a guide wire contained within a lumen of the catheter. Pacing conditioning sequences are then initiated when catheter is introduced into artery to providing global cardiac conditioning. The pacing may be continued throughout procedure. The dilitation balloon (or other revascularization instrument) is then positioned next to the target lesion, and cryotherapy is initiated just prior to reperfusion using the heat sink incorporated into the catheter. The lesion is dilated and, in one embodiment, a stent is deployed in the artery. The cryotherapy may be continued for a short period of time subsequent to reperfusion and then discontinued. For example 20-30 seconds of cryotherapy is usually sufficient to elicit the desired benefit. Finally, the pacing conditioning discontinued, and the catheter is removed.

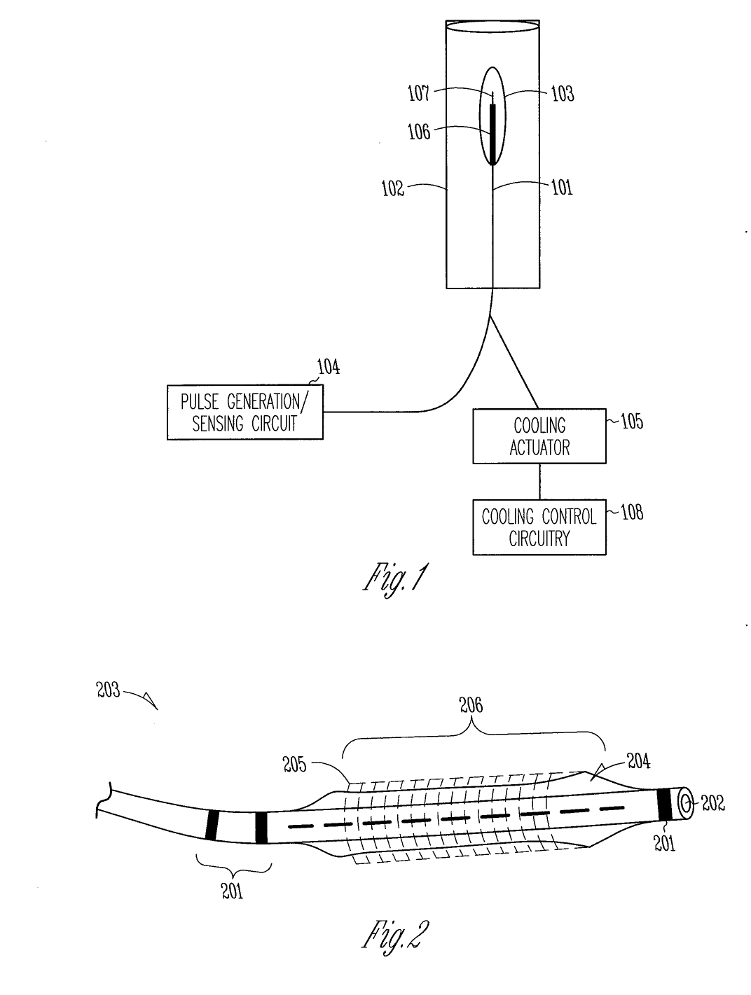

FIG. 1 is a diagram of an apparatus for performing the procedure as just described. The catheter 101 is shown as being disposed within a blood vessel 102 at a target location. A balloon actuator pressurizes and inflates an angioplasty balloon 103 to dilate the lesion (and possibly deploy a stent). Pulse generation/sensing circuitry 104 is connected to one or more pacing electrodes of the catheter via one or more conductors within the catheter. The pulse generation circuitry may be configurable to deliver the pacing in either an asynchronous or synchronous mode, where in the latter case the pacing electrode is also used to sense intrinsic cardiac activity. When pacing in a synchronous mode, the escape interval should be set below the patient's intrinsic heart rate to ensure that the pacing is sufficient to elicit the desired conditioning effect. Cooling actuator 105 is connected to the heat sink 106 of the catheter and, depending upon the embodiment, may be an electrical source connected to the heat sink via a conductor incorporated into the catheter or may be a pumping mechanism for channeling coolant into the catheter. The cooling control circuitry 108 controls the operation of the cooling actuator to activate the heat sink and cause cooling of the arterial walls prior to and subsequent to dilation. The cooling control circuitry may also be connected to a temperature sensor 107 incorporated into the catheter for sensing the temperature of the tissue cooled by the heat sink. The cooling control circuitry may then be configured for controlling the operation of the heat sink in accordance with the temperature sensed by the temperature sensor.

FIG. 2 illustrates an exemplary embodiment of a balloon catheter configured to deliver pacing therapy and cryotherapy. The catheter 201 is shown as being fitted over a guide wire 202. The catheter is equipped with one or more pacing electrodes 203, shown in the figure as being ring-type electrodes on the surface of the catheter at its distal end. Other types and designs of electrodes could also be used. The electrodes are connected to pulse generation/sensing circuitry by conductors within the catheter. An angioplasty balloon 204 inflatable by pressurized fluid is also shown along with a stent 205 deployable by the balloon. A heat sink 206 is incorporated into the catheter and acts to draw heat energy from the balloon when the latter is in contact with the arterial lesion.

The invention has been described in conjunction with the foregoing specific embodiments. It should be appreciated that those embodiments may also be combined in any manner considered to be advantageous. The embodiments described herein may also be combined with any of the devices or methods described in the above-referenced '828 application. Also, many alternatives, variations, and modifications will be apparent to those of ordinary skill in the art. Other such alternatives, variations, and modifications are intended to fall within the scope of the following appended claims.

What is claimed is:

1. An apparatus, comprising:
a catheter adapted for insertion into a coronary artery;
an instrument at the distal portion of the catheter for performing a coronary revascularization procedure;
a pacing electrode incorporated into the distal portion of the catheter;
a conductor within the catheter for connecting the pacing electrode to pulse generation circuitry; and,
a heat sink incorporated into the distal portion the catheter for cooling vascular tissue adjacent the catheter.

2. The apparatus of claim 1 wherein the instrument for performing a coronary revascularization procedure is an inflatable angioplasty balloon.

3. The apparatus of claim 2 further comprising a stent deployed by the balloon.

4. The apparatus of claim 3 wherein the stent is employed as the pacing electrode.

5. The apparatus of claim 1 wherein the heat sink is a thermoelectric cooling module and further comprising a conductor incorporated into the catheter for transmitting power to the thermoelectric cooling module.

6. The apparatus of claim 1 wherein the heat sink is a zirconate titanate module and further comprising a conductor incorporated into the catheter for electrically activating the zirconate titanate.

7. The apparatus of claim 1 further comprising pulse generation circuitry configured to deliver pacing pulses in an asynchronous pacing mode.

8. The apparatus of claim 1 further comprising pulse generation and sensing circuitry configured to deliver pacing pulses in a synchronous pacing mode.

9. The apparatus of claim 1 further comprising a temperature sensor incorporated into the catheter for sensing the temperature of the tissue cooled by the heat sink.

10. The apparatus of claim 9 further comprising control circuitry for controlling the operation of the heat sink in accordance with the temperature sensed by the temperature sensor.

11. A method, comprising:
disposing a catheter into a coronary artery;
using the catheter to perform a coronary revascularization procedure;
delivering cardiac pacing therapy via a pacing electrode incorporated into the catheter; and,
delivering cryotherapy to cool vascular tissue adjacent the catheter via a heat sink incorporated into catheter.

12. The method of claim 11 wherein the coronary revascularization procedure is balloon angioplasty.

13. The method of claim 12 further comprising deploying a stent within the coronary artery.

14. The method of claim 13 further comprising utilizing the stent as the pacing electrode.

15. The method of claim 11 wherein the heat sink is a thermoelectric cooling module.

16. The method of claim 11 wherein the heat sink is a zirconate titanate module.

17. The method of claim 11 further comprising pulse generation circuitry configured to deliver pacing pulses in an asynchronous pacing mode.

18. The method of claim 11 further comprising delivering pacing pulses in a synchronous pacing mode.

19. The method of claim 11 further comprising sensing the temperature of the tissue cooled by the heat sink via a temperature sensor incorporated into the catheter.

20. The method of claim 19 further comprising controlling the operation of the heat sink in accordance with the temperature sensed by the temperature sensor.
